(19) United States
(12) Patent Application Publication
(10) Pub. No.: US 2014/0187993 A1
(43) Pub. Date: Jul. 3, 2014

(54) BREATH ALCOHOL RECORDING AND TRANSMISSION SYSTEM

(71) Applicant: Sentinel Offender Services, Irvine, CA (US)

(72) Inventors: Robert Contestabile, Cota de Caza, CA (US); Steven Durana, San Juan Capistrano, CA (US)

(73) Assignee: SENTINEL OFFENDER SERVICES, Irvine, CA (US)

(21) Appl. No.: 14/139,993

(22) Filed: Dec. 24, 2013

Related U.S. Application Data
(60) Provisional application No. 61/746,334, filed on Dec. 27, 2012.

(51) Int. Cl. A61B 5/08; A61B 5/00

(52) U.S. Cl. CPC A61B 5/082; A61B 5/742; A61B 5/486; A61B 5/0004; A61B 5/0022
USPC 600/532

(57) ABSTRACT

The present invention provides a portable alcohol tester with biometric and location recordation and transmission. The portable unit may use photo, retina, or fingerprint recordation while the system is testing to ensure the identity of the participant. The unit may also use Wi-Fi stations, cellular towers or global position satellites to identify a location, time, and date for the test and record said information. The unit may be equipped with RF, Wireless, cellular, or Bluetooth™ communications to transmit the test information to a remote data center. The data center is equipped to merge the various different data points received into a coordinated presentation. The test information can be used by criminal justice agencies for recording the time, date, location, biometric ID, and breath alcohol content. The same information could be used for participant monitoring or rehab institutions.

### CROSS-REFERENCE TO RELATED APPLICATIONS

This application claims priority to provisional application No. 61/746,334 filed Dec. 27, 2012 and titled BREATH ALCOHOL RECORDING AND TRANSMISSION SYSTEM

### BACKGROUND OF THE INVENTION

The present invention generally relates to a breath alcohol tester. More particularly, the present invention relates to a portable breath alcohol tester with recordation of biometric and location information and the ability to transmit the information to a local display and remote data center.

Standard breath alcohol testers are well known in the field, and are used to give criminal justice agencies and general persons information relating to the approximate blood alcohol content based on alcohol in the participant's breath. The participant blows into the unit and the device determines and displays the information on a local display.

Embodiments of the prior art were limited in giving only a read out that the officer would have to record for use later. Further there is no method of confirming the subject or location of the test. The court and participant monitoring services must rely on the accuracy of the officer's report over the participant's testimony.

In other embodiments of the prior art, the alcohol testing device would transmit the data to a computer system which then compiles data over the course of several hours or days. After being compiled, the information is sent to criminal justice agent (e.g., probation officer, police officer) or treatment personnel for review. This lack of real-time reporting is problematic in that it indicates only that a participant had been drinking; it did not indicate that participant was currently drinking, or where the participant could be located.

In other embodiments of the prior art, breath alcohol testers have been used for interlock devices, which prevent cars from starting unless the driver first ‘blows’ less than a predetermined breath alcohol content. One weakness of prior art devices is that, because no biometric data is collected, they could be defeated by having a person other than the driver perform the test.

The use of transdermal alcohol monitoring for participant monitoring or rehab programs has been used by attaching an irremovable detector to the participant that monitors the alcohol level and records the information for later retrieval. This requires the user to have a bulky, unattractive and attention drawing unit attached to them at all times. Additionally, the transdermal bracelets have demonstrated a propensity to induce dermatitis and sores requiring periodic shifting of the device between ankles.

### BRIEF SUMMARY OF THE INVENTION

One or more of the embodiments of the present invention provide a portable alcohol tester with biometric and location recordation and transmission. The portable unit may use photo, retina, or fingerprint recordation while the system is testing to ensure the identity of the participant. The unit may also use Wi-Fi stations, cellular towers or global position satellites to identify a location, time, and date for the test and record said information. The unit may be equipped with RF, Wireless, cellular, or Bluetooth™ communications to transmit the test information to a remote data center. The data center is equipped to merge the various different data points received into a coordinated presentation. The test information can be used by criminal justice agencies for recording the time, date, location, biometric ID, and breath alcohol content. The same information could be used for participant monitoring or rehab institutions.

### BRIEF DESCRIPTION OF THE DRAWINGS

FIG. 1 illustrates a block diagram schematic representation of breath alcohol tester according to an embodiment of the present invention.

FIG. 2 illustrates an outer appearance of an embodiment of the Breath alcohol tester.

### DETAILED DESCRIPTION OF THE INVENTION

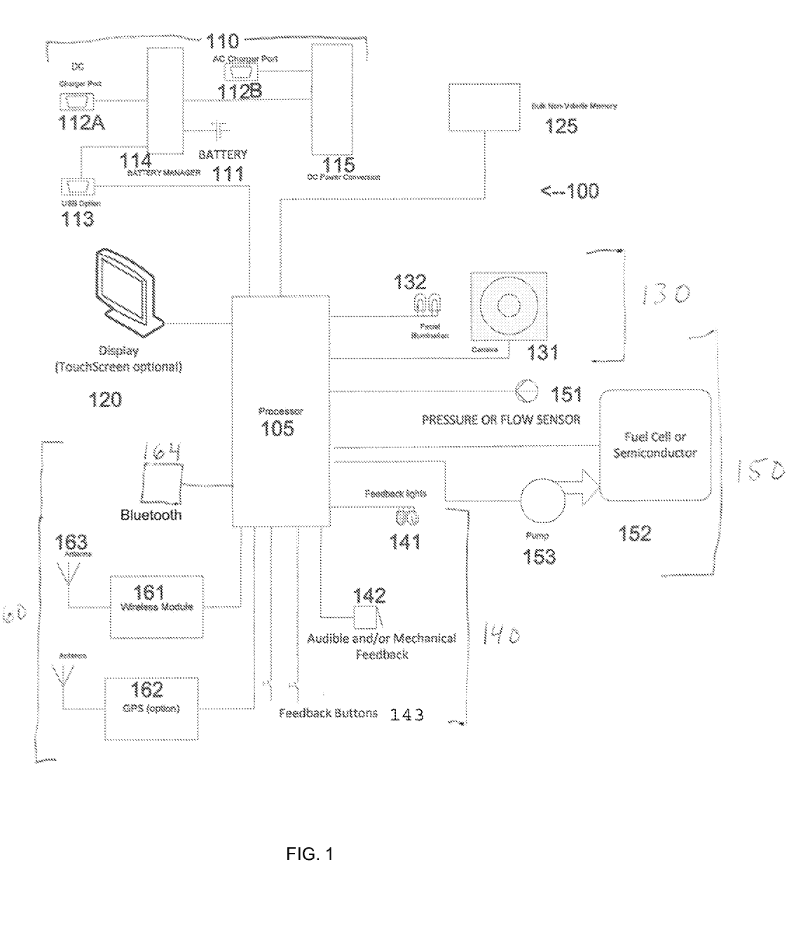

FIG. 1 illustrates a breath alcohol tester 100 according to an embodiment of the present invention. The breath alcohol tester 100 includes a processor 105, a power supply 110, a display 120, a memory unit 125, a biometric recorder 130, a feedback unit 140, a testing unit 150, and a communications unit 160. The power supply 110 includes a battery 111, a battery manager circuit 114, a dc power charger port 112A, an AC power charger port 112B, a DC power converter 115 and a USB port 113. The biometric recorder unit 130 includes a camera 131 and a facial illuminator 132. The feedback unit 140 includes feedback lights 141, audio or mechanical feedback unit 142, and feedback buttons 143. The testing unit includes a pressure sensor 151, a fuel cell 152 and a pump 153. The communications unit 160 includes a wireless module 161 and an antenna 163.

The AC power charger port 112B, of the power supply 110, is electrically connected to the DC power converter 115. The DC power converter 115 is electrically connected to the battery manager circuit 114 and all the circuitry requiring conditioned DC power. The DC power converter 115 receives electrical power from the AC Charger Port 112B and the Battery Manager 114 and produces regulated voltages with sufficient current capacity to energize the remaining electrical components. The battery management circuit is electrically connected to the DC power charger port 112A, the battery 111, the USB port 113, and in parallel with the USB port to the processor 105. The processor 105 is in data/electrical communication with the display 120, the memory unit 125, the facial illuminator 132, the camera 131, the pressure sensor 151, the fuel cell 152, the pump 153, the feedback lights 141, the audio/mechanical feedback 142, the feedback buttons 143, and the wireless module 161. The pump 153 is in fluid contact with the fuel cell 152. The wireless module is in data/electrical communication with the antenna 163.

In operation, the AC power charger port 112B, if provided, is connected to a standard wall outlet or a wall power adapter providing low voltage AC power. AC power is supplied by the AC power charger port 112B to the DC power converter 115. The DC power converter 115 rectifies and transforms the AC to DC power at the desired voltage. The DC power converter 115 receives electrical power from the AC Charger Port 112B and the Battery Manager 114 and produces regulated voltages with sufficient current capacity to energize the remaining electrical components. The battery manager 114 supplies power to maintain the charge on the battery 111 in a regulated manner to maximize battery life. The DC power charger port 112A is connected to a standard wall outlet with a DC power supply circuit. The DC power charger port 112A supplies the Battery manager circuit 114 and the DC Power Converter 115 with DC power. The USB port 113 supplies DC power to the battery manager and the DC Power Converter 115 circuit through a USB connector. The DC Power Converter 115 supplies electrical power to the processor 105.

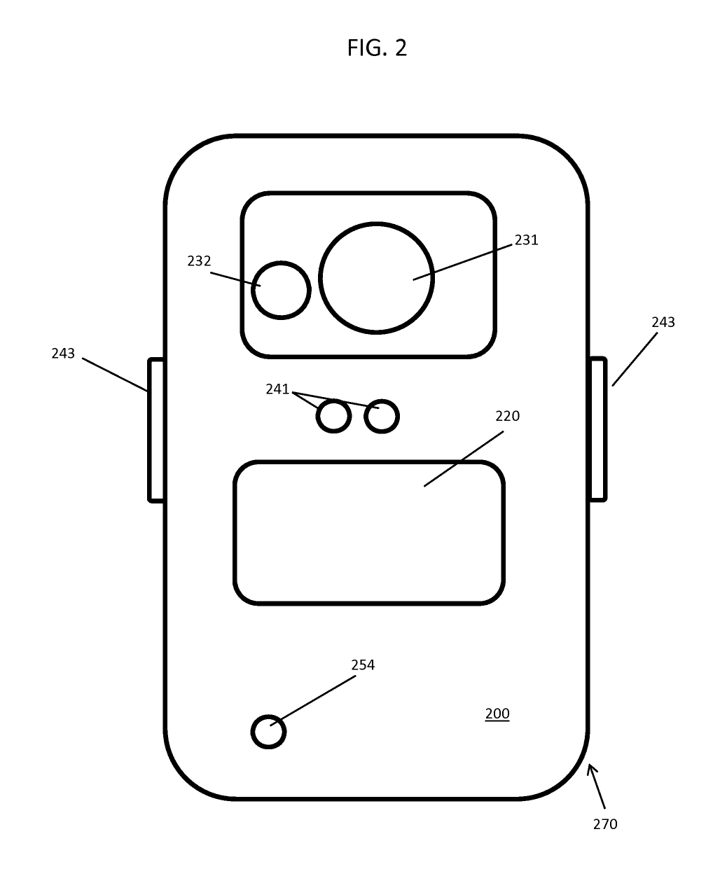

The feedback buttons 143 are used to change modes, display, and start the test. The feedback buttons also are used to acknowledge receipt of messages initiated by the officer or agency which appear on the Display 120. The user depresses the feedback button or buttons 143 to start the test, sending a signal to the processor 105 to execute a test. The processor sends a signal to the feedback lights 141 to energize the red light. The feedback lights 141 including one or more light emitting diodes or various colors. The user blows into the input straw, such as 254 of FIG. 2. When the pressure flow sensor 151 detects pressure above a predetermined level for a sufficient period of time to insure that a deep lung sample has been acquired it sends a signal to the processor 105. The processor de-energizes the red and energizes the green feedback light 141, indicating to the user that they are blowing into the sample tube at an acceptable volume. The processor also energizes the pump to send the volume of user's breath at a predetermined rate to the fuel cell 152. In some embodiments the fuel cell 152 is a semiconductor sensor. When a predetermined volume of breath has been processed by the fuel cell 152, the processor sends a signal to the pump to de-energize, and a signal to the feedback lights to indicate that the test is complete, such as flashing both red and green lights. The fuel cell 152 measures the level of alcohol in the sample and sends the results to the processor 105. The processor (optionally) sends the results to the display 120 for the user to view. The capability to display the test results to the client is optionally configurable by the officer or agency.

During the test the camera 131 records biometric data, such as a picture of the participant's face. The camera 131 should be of sufficient quality to allow for a detailed image, such as six megapixels. The processor 105 sends a signal to the facial illuminator 132 to energize to provide adequate light for a picture of the participant. The facial illuminators 132 include one or more light emitting diodes. The processor then sends a signal to the camera 131 to capture an image of the participant. The camera sends the image data to the processor 105. The processor 105 sends the test results, received from the fuel cell 152, as well as date/time information, and the image data to the memory unit 125. The memory unit 125 can be any digital memory device, but is preferred to be solid state memory. The memory unit 125 stores the recorded information until retrieved.

The data stored in the memory unit 125 may be retrieved by plugging a computing device into the USB port 113. The computing device sends a signal to the processor 105 to retrieve the recorded data. The processor 105 sends a request for the recorded data to the memory unit 125. The memory unit 125 sends the recorded data to the processor 105, which sends the data to the computing device through the USB port 113. After the test results are received, the processor receives commands and configuration data from the computing device.

Additionally the data stored in the memory unit may be sent to a remote database by the wireless module 161. When a record has been sent to the memory unit or at a predetermined time interval the processor 105 will send a signal to the wireless module to verify data signal connection. The wireless module 161 detects the data signal strength, using the antenna 163, and sends the information to the processor 105. If a data signal is present the processor 105 request the wireless module to establish a communications link using the antenna 163. The processor 105 requests the recorded data from the memory unit 125 and sends it to the wireless module. The wireless module 161 transmits the recorded data to the remote database through the antenna 163. Alternatively, the wireless module may be a cellular module and perform the operation in the same method. In the event that the device is out of range of wireless or cellular communication, the memory unit 125 will store the data until such time that communication is restored. After the test results are received, the processor receives commands and configuration data from the host computer.

An embodiment of the breath alcohol tester includes a Bluetooth™ or Wi-Fi module 164. The Bluetooth™ or Wi-Fi module 164 is in data/electrical communication with the processor 105. The Bluetooth™ or Wi-Fi module 164 transmits the recorded data to a relay unit which is connected to a phone line or the internet. The relay unit sends the recorded data to the remote database.

An embodiment of the breath alcohol tester includes a global position detector 162. The global position detector 162 is in electrical connection with the processor 105. During a test the global position detector uses satellite positioning to determine a global position. The global position detector 162 sends the global location data to the processor 105. The processor 105 includes the global position data in the recorded data and stores it in the memory unit 125. If the unit is equipped with a cellular module 163, the module may be used to triangulate a global position using cellular towers. The location data would be sent to the processor 105 and stored as discussed.

The test information can be reviewed and stored at remote locations for evidentiary purposes in criminal justice proceedings. Additionally, the test data can be used to aid and monitor persons in rehab of monitoring programs.

In one embodiment the date/time information is generated by an onboard clock. This data may be superseded or updated by a GPS signal form the global position detector 162 with date/time data or a cellular signal from the wireless module 161 with date/time data or from the host computer or computing device.

In one embodiment of the breath alcohol tester, the feedback buttons 143 serve a second purpose of recording biometric data. When this feature is equipped one or more of the feedback buttons 143 must be depressed during the test. The feedback buttons are equipped with biometric fingerprint scanners. The processor 105 sends a signal during the test to the feedback button to execute a biometric scan. The feedback buttons use the fingerprint scanner to collect an image of the participant's fingerprint. The feedback buttons 143 sends the fingerprint data to the processor 105. The processor 105 stores the fingerprint data with the recorded data for the test in the memory unit 125.

In an embodiment of the breath alcohol tester the camera 131 is designed and oriented to capture a retinal image. The retinal image stored in the same manner as the facial image data. The use of retinal image provides increased reliability in the identity of the test participant.

In an embodiment of the breath alcohol tester the display 120 is a touch screen and the user may perform operations and selections from the screen or with the feedback buttons. In other embodiments the feedback buttons are removed and all functions are controlled by the touch screen.

An embodiment of the invention includes an audio and/or mechanical feedback unit 142. When a test starts or ends the audio and/or mechanical unit 142 may shake or rattle to alert or inform the user of the change in status. If the participant is required to perform a test at a specified time or interval the processor may send a signal to the audio and/or mechanical feedback unit 142 to actuate physically or with a beep or similar audio cue. If the unit is equipped with speaker the processor could send a signal to the audio and/or mechanical unit 142 to speak instructions and results.

FIG. 2 illustrates an embodiment of the exterior of the breath alcohol tester 200. The breath alcohol tester includes a display 220, a camera 231, a facial illuminator 232, feedback lights 241, an input straw 254, and a case 270. The case 270 houses the camera 231, the facial illuminator 232, the feedback lights 241, and the input straw connection 254.

In operation the participant inserts a disposable sample straw into the connection for the input straw 254. The user then depresses one or more of the feedback buttons 243 to start the test. The red feedback light 241 energizes to inform the participant that the test has begun and to blow into the sample straw. When the participant has blown with adequate force the green feedback light 241 will energize and the red will de-energize. The use in color of the feedback lights 241 is illustrative only and any combination may be used. The facial illuminator 232 will energize to light the face of the participant, and the camera 231 will capture an image of the participant. When the test is complete both the red and green feedback lights 241 will flash to inform the user that the test is complete. The breath alcohol level of the participant will (optionally) appear on the display 220.

Prior portable breath alcohol devices merely took a sample and then displayed the results via a numeric digital display. Results were not date and timestamped, nor were they correlated with a biometric data point such as a photograph of the participant being tested. In addition, results were not communicated wirelessly to a central monitoring system for further plotting on maps with availability to be ‘alarmed’ and redistributed to field officers. Another drawback of prior devices is that reporting was delayed by hours, or sometimes days. This delay can lead to difficulty in proving that the participant was or was not in compliance with terms of probation or other legal restrictions imposed on the participant.

In one embodiment, the system can be used by criminal justice agent in the field. When a participant is identified in the field by a government officer or other authorized officer as requiring a breath alcohol test, the officer, using the breath alcohol tester, supervises the collection of a breath alcohol test by instructing the participant to blow through a small straw into the GPS-enabled breath alcohol tester. In other embodiments, the system is designed to be carried by the participant, who is alerted by the device that a test is required. In this case, the participant operates the device himself by blowing through a small straw into the breath alcohol tester.

After enough breath is collected an LED light flashes green while simultaneously taking a picture of the participant via a camera with sufficient clarity, such as 6 mega pixels. The collected breath alcohol sample is pressurized and analyzed using fuel cell extract technology. The results of the test, the picture of the participant (or other biometric data), the GPS location data point and the date and time are then communicated via wireless data to a central monitoring system. The monitoring system then plots the data on a mapping system and further distributes the results of the tests to other officials remotely as required. Mapping system data can include Google™ street level information which can be used to verify the location of the test such as a bar or other public place.

In a preferred embodiment, the data points described above are collected and processed by a central monitoring system. A central monitoring system is used to manage the different expectations and needs of the various users of the breath alcohol recording and transmission system. In practice, the type of system described herein may be used by hundreds or thousands of different government agencies, rehabilitation centers, and the like. Each of these entities may desire to have the monitoring processed in a slightly different way. Thus, the central monitoring system must be highly flexible in how it applies the rules set by the requesting entity. Each entity creates a profile or monitoring template describing with particularity the information it requires from the participant, the schedule on which the information is collected, and how and to whom that information is reported after collection. In one embodiment, the central monitoring system also allows the entities to change the parameters of the monitoring template as needed by remote access to the database, as through a secure internet portal. This allows greater flexibility in monitoring based on the agency's needs, and eliminates the need to involve additional personnel to effect changes, thus saving time and money to the user and the central monitoring system provider.

The entities that use the system described herein are provided a user interface that integrates the various data points collected by the system. The central monitoring system monitors various field monitoring systems (photograph of the participant, fuel cell readings, time, date, GPS location, etc.). All of these various systems report their data through to the central monitoring system, or online transaction processing system. The central monitoring system then checks this data against the parameters set by the requesting entity and determines how the data should be processed. The data is then presented on what appears to be a single user interface. In reality each type of data is likely to run on a different database or different system architecture. It is an object of this invention to unify the data for presentation in a single interface.

Each government agency or entity requires specific actions to occur in response to the various transactions received and processed through the central monitoring system. Many of the transactions do not require operator reaction. Such ‘no reaction’ transactions are simply filed for review or reporting during the normal course of business. Other transactions require various levels of response from operators or are timed and watched for additional operator action. Some transactions are immediately marked as alarms and sent via text to government agency officials. In some cases, the alarms may be subsequently nullified by events received from the various field devices. Alarm transactions may require that a monitoring center operator take some type of action such as calling an offender's home, calling an officer on the telephone or calling the local police and reporting an activity. In practice, the breath alcohol testing and monitoring system provides the exact information needed to the government agency or other entity with little or negligible delay. Thus the information does not merely pass through a central computer, but is compiled and adapted to the particular needs of the agency.

In one embodiment of the invention, multiple users of the breath alcohol recording and transmission system are enabled to view the reporting information collected. In this case, the term “user” includes the participant being tested, the government agency (or agencies) and personnel charged with monitoring the participant, rehabilitation centers treating the participant, court personnel, and the like. In a preferred embodiment, the information is immediately pushed to a portable device such as a smart phone, tablet, or laptop for viewing by the user. A user can also view the reports either by accessing a particular website hosting the information, through notifications sent to a smart phone, laptop, or similar device, or through a periodic report sent by email, facsimile, mail or other means to a requesting user. Because the information collected is a compilation of several layers of data, it also provides for a more sure evidentiary basis, in the event that the data is to be used in court proceedings.

In effect, the monitoring system described here notifies local criminal justice agencies in real time whether a participant is in compliance with the terms of his probation or rehabilitation goals. In the case that the participant is not in compliance (i.e., the test shows an elevated level of alcohol in the participant), officers of the relevant criminal justice agency are notified at that moment that the participant is in violation, and can also be told exactly where the participant is at the moment. By so doing, the integration of the portable device described above with the central monitoring system is a substantial improvement over the prior art, which could not provide the benefits of real-time monitoring with the surety of identifying biometric information and location information.

The breath alcohol tester's relatively small size and portability make it easy to carry and use in the field. The size and portability also make it much less noticeable to other persons, because it is not continuously worn. The participant may discreetly perform the test and replace the system into a bag or other transportation device without drawing attention to the use. The self-reporting system allows for the test data to be sent to monitoring centers without the participant performing further operations. Although the test data may be retrieved manually, it is not required for the monitoring service or the participant to travel to the other to retrieve the data increasing convenience for both.

Because the device can be carried by the participant, and because the government agency is allowed to access the data or the monitoring profile at any time, it is also possible to manually trigger a testing event remotely. For example, if a criminal justice agent receives information that the participant is likely to be in violation of his probation, the officer can signal the participant's device to create an audible or tactile alarm via the feedback unit 142 indicating that the participant is to blow into the device immediately. The information can then be sent immediately to the officer to verify whether the participant is in compliance with terms of his probation. Alternatively, the officer may send additional instructions to the participant, which instructions are displayed on the device in real time. The participant, upon receiving a manually-triggered request for testing may acknowledge that the request was received and understood by pressing a button, such as the feedback button 143.

Alternatively, the criminal justice agency or treatment provider may choose to create periodic testing schedules for the participant. In such a case, the criminal justice agent could create a new profile via a secure web portal. This new profile would be communicated to the device, which would then be updated with the new schedule. As a practical example of this use, a probation officer could determine that a participant is to blow into the tester at 10:05 A.M. every day during the coming week. The time to take the test may also be adjusted randomly by the unit itself but within some nominal limits. The unit may also be instructed to demand tests based upon a completely random schedule or a random schedule but within specific time frames. The minimum interval between tests may also be specified. The probation officer would create this schedule using the profile described above. The new profile is then transmitted via cellular, Bluetooth™, USB or other similar transmission method to the participant's device. In a preferred embodiment, the participant would not be informed of the new test schedule, but would be alerted via audible or tactile cue at the newly appointed time to blow into the tester (in this case at 10:05 A.M.). In this example, the probation officer may alternatively choose to test at different times each day of the week, or may even update the schedule every day, week, or month, as desired. Using this method, the probation officer could further ensure compliance with probation conditions through surprise or seemingly random test times.

An added feature of the invention is the ability for the criminal justice agency or treatment provider to communicate via personalized text messages directly with the participant. The invention offers the ability to send SMS messages or similar direct messages directly to the participant and alerts the individual with an audible signal. The message appears on the LED screen and the participant acknowledges receipt of the message.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. A portable device for breath alcohol testing and monitoring, comprising:
a processor, a power supply, a display, a memory unit, a biometric recorder, a feedback unit, a testing unit, and a communications unit;
wherein, the portable device records biometric information and location information of a user;
wherein the portable device transmits the information to said display and to a remote data center; and
wherein, said location information is verified by wireless communication.

2. The portable device of claim 1, wherein the biometric information comprises breath alcohol content.

3. The portable device of claim 2, wherein the location information is verified by use of global positioning satellite systems.
